(19) United States
(12) Patent Application Publication
(10) Pub. No.: US 2012/0201923 A1
(43) Pub. Date: Aug. 9, 2012

(54) SYNTHETIC PHYTASE VARIANTS

(75) Inventors: Stefan Haefner, Speyer (DE); Serwe Annegret, Ludwigshafen (DE); Beata Stosik, Mannheim (DE)

(73) Assignee: BASF SE, Ludwigshafen (DE)

(21) Appl. No.: 13/502,647

(22) PCT Filed: Oct. 18, 2010
(86) PCT No.: PCT/EP10/65624

§ 371 Date: Apr. 18, 2012

(30) Foreign Application Priority Data
Oct. 22, 2009 (EP) 09173782.5

(51) Int. Cl. C12N 9/16; C12N 15/63; A23K 1/165; C12N 1/19; C12N 1/21; C12N 15/55; C12N 1/15

(52) U.S. Cl. 426/2; 435/196; 536/23.2; 435/320.1; 435/254.3; 435/254.23; 435/254.6; 435/254.21; 435/252.31; 435/252.33; 435/254.2; 426/61

(57) ABSTRACT

The invention relates to a synthetic phytase having an increased thermal stability and also to an isolated nucleic acid sequence encoding a synthetic phytase, and the use of the phytase in an animal feed for reducing the phosphtate content in manure, and also animal feed additives and animal feeds comprising the synthetic phytase.

The present invention relates to phytases, amino acid sequences encoding phytase enzymes and also nucleotide sequences which encode phytases and methods for producing and using phytases, and also animal feeds comprising these phytases.

Phosphorus is an essential element for the growth of living organisms. In animal production, feed must generally be supplemented with inorganic phosphorus in order to achieve good growth performances. In cereals and legumes, phosphorus is principally stored in the form of phytate. However, monogastric animals such as pigs, poultry and fish are not able to absorb phytate or phytic acid directly, and so this leads to phytate excretion which means overenrichment with phosphorus in regions in which intensive farm animal production takes place. In addition, phytic acid, by binding metals such as calcium, copper or zinc, acts as a composition adversely affecting metabolism in monogastric animals. In order to compensate for the phosphate deficit of these animals and to ensure adequate growth and adequate health, inorganic phosphate is added to the animal feed. This addition of inorganic phosphate is expensive and leads to a further pollution of the environment. By using a phytase in animal feeds, the phytate is hydrolyzed and yields a lower content of inositol phosphate and inorganic phosphates in the manure. The addition of phytases to animal feeds improves the availability of organic phosphorus and reduces the environmental pollution caused by excreted phosphates bound to phytate. In the literature, a great number of natural phytases are described, not only of fungal but also of bacterial origin.

Phytases, also called myo-inositol hexakisphosphate phosphohydrolases, are a class of phosphatases which are able to release at least one phosphate group from phytate.

EP 420 358 describes in general the cloning and expression of microbial phytases, WO 2006/38062 describes microbial phytases which originate from Citrobacter freundii as an addition to animal feeds, WO 2007/112739 describes phytases based on a natural phytase from Citrobacter braakii, and also methods for production thereof and use thereof in animal feeds.

In Haefner et al. (Haefner S., Knietsch A., Scholten E., Braun J., Lohscheidt M. and Zelder O. (2005) Biotechnological production and application of phytases. Appl Microbiol Biotechnol 68:588-597), a multiplicity of known uses of phytases in the sector of human or animal nutrition are described. Further uses of phytases such as, for example, the use for hydrolyzing biomass or starch in bioethanol production is described in WO2008/097620.

WO 2008/116878 describes a phytase from Hafnia alvei and its protein sequence. Zinin et al. (FEMS Microbiology Letters (2004) 236:283-290) disclose a phytase from Obesumbacterium proteus, the sequence of which is deposited in the UNIPROT database having the accession number Q6U677. The patent applications WO 2006/043178, WO 2008/097619 and WO 2008/092901 describe phytases from various Buttiauxella sp. Natural phytases having the currently highest specific activities include the natural phytases from Yersinia intermedia (WO2007/128160) and Yersinia pestis (WO02/048332).

However, none of these currently available phytases display those properties which are necessary for producing animal feed additives. The currently available phytases do not exhibit sufficient thermal stability in order to be used in the production of animal feed pellets without considerable loss of the activity thereof. During the production of animal feed pellets, the phytase is pressed together with further customary animal feed components under high temperatures and moisture in order to be fed as a whole to the farm animals. During this hot and moist pressing, considerable losses of phytase activity occur. A possibility of preventing this loss in activity is the complex coating of phytase particles, so that they are protected against the action of heat. This coating of the phytase additives causes considerable additional costs due to the fats or polymers employed which are used for the coating.

An object of the present invention was therefore to provide a phytase which has a sufficient thermal stability such that it can be used in the production of feed pellets without additional protective measures such as coating. It was a further object of the present invention to provide a phytase having a sufficiently high specific activity such that the amount of phytase to be used overall during the feed production is as low as possible. A further object of the invention was to provide a phytase which can be used over a broad pH range, in order to be usable in the differing pH ranges of the digestive tracts of differing animal species and to retain the activity thereof even in the event of variations of the pH range due to varying feed components.

These objects are achieved by a synthetic phytase which has at least 70% identity with the amino acid sequence of SEQ ID 18. These phytases according to the invention have a temperature optimum of at least 63° C. and a thermal stability of at least 65° C. and are therefore suitable to be used in the production of feed pellets without their suffering a considerable loss of their activity due to the hot and moist conditions during pelleting. In addition, they have a broad pH range of above 3 pH units in which they retain at least 50% of their relative activity, and so they can be used in a multiplicity of animals and together with differing feed constituents without losses in the activity thereof and thereby increased excretion of the phosphate by the animals occurring. Preferably, the synthetic phytase according to the invention has at least 75%, preferably 80%, particularly preferably 85%, and preferably 90, 91, 92, 93, 94, 95, 96, 97, 98, 99%, identity with the amino acid sequence of SEQ ID 18.

The identity between two protein sequences or nucleic acid sequences is defined as that calculated by the program needle in the version available in October 2009. Needle is a part of the freely available EMBOSS program package which can be downloaded from the website http://emboss.sourceforge.net/. The standard parameters are used: gap open 10.0 (“gap open penalty”), gap extend 0.5 (“gap extension penalty”), data file EBLOSUM62 (matrix) in the case of protein and data file EDNAFULL (matrix) in the case of DNA.

According to a particular embodiment, the synthetic phytase has a change of the amino acid at at least one of the positions selected from the group consisting of position 1, 6, 12, 17, 84, 89, 92, 109, 137, 138, 140, 142, 143, 149, 156, 202, 205, 207, 208, 209, 228, 234, 243, 247, 248, 251, 255, 256, 261, 270, 304, 314, 320, 349, 356, 373, 382, 399, 402 and 413, based on the position according to SEQ ID 18. A change, in the context of the present invention, is taken to mean replacement of the original amino acid as specified in SEQ ID 18 in the sequence protocol by another amino acid. The amino acids in this case are named by the conventional one-letter code. By changing one or more amino acids it is possible to increase further the thermal stability of the synthetic phytase or to broaden the optimum pH range or to increase the specific activity.

Advantageously, the synthetic phytase has at least 5 changes in the amino acid sequence based on SEQ ID 18, in particular it has at least 10, at least 12, at least 14, at least 16, at least 17, at least 18, at least 19, and very particularly preferably at least 20, changes.

Preferably, at least one of the amino acids at one of the positions selected from the positions 1, 6, 12, 17, 84, 89, 92, 109, 137, 138, 140, 142, 143, 149, 156, 202, 205, 207, 208, 209, 228, 234, 243, 247, 248, 251, 255, 256, 261, 270, 304, 314, 320, 349, 356, 373, 382, 399, 402 and 413, based on the position in SEQ ID 18, is replaced by one of the following amino acids, wherein, advantageously, the newly introduced amino acid is, at position 1 N, D, Q, H, at position 6 V, I, L, T, S, at position 12 N, D, Q, H, S, at position 17 N, D, Q, H, at position 84 V, I, L, T, S, at position 89 T, S, V, at position 92 E, Q, K, R, N, P, A, S, G, H, at position 109 K, R, E, D, at position 137 L, I, V, at position 138 N, Q, H, S, at position 140 P, A, N, at position 142 T, S, V, at position 143 Y, F, W, at position 149 H, N, S, at position 156 R, K, E, D, at position 202 S, T, A, V, at position 205 R, K, E, D, at position 207 E, Q, D, R, N, T, S, V, A, I, L, M, at position 208 M, L, I, at position 209 S, T, at position 228 Y, F, W, at position 234 N, D, Q, H, S, I, V, L, M, at position 243 K, R, D, at position 247 K, R, E, at position 248 L, I, M, V, at position 251 N, D, Q, H, S, I, V, L, M, at position 255 V, I, L, T, S, at position 256 Y, F, W, H, N, S, at position 261 E, Q, D, K, R, N, at position 270 K, R, E, D, at position 304 V, I, L, T, S, at position 314 G, A, at position 320 L, I, M, V, at position 349 R, K, E, D, at position 356 L, I, M, V, at position 373 I, V, L, M, at position 382 G, A, at position 399 I, V, L, M, at position 402 N, D, Q, H, S and at position 413 L, I, M, V, Q, E, N, K, R.

In a preferred embodiment, the synthetic phytase has at least one of the following changes with respect to the amino acid sequence of SEQ ID 18: SIN; A6V; K12N; S17N; A84V; A89T; D92E; D92P; D92A; D92N; Q109K; M137L; D138N; S140P; A142T; H143Y; Q149H; T156R; N202S; N202T; G205R; K207E; K207T; K2071; V208M; A209S; H228Y; K234N; K2341; E243K; D247K; S248L; K251N; K2511; A255V; Q256Y; Q256H; S261E; N270K; A304V; S314G; T320L; Q349R; F356L; S3731; E382G; T3991; K402N; H413L; H413Q.

In this list the amino acid from SEQ ID 18 mentioned before the respective position number is replaced by one of the amino acids mentioned after the position number. Here, any possible mentioned amino acid replacement with any of the remaining changes is possible in combination.

Advantageously, the synthetic phytase of the present invention comprises at least 5 of the abovementioned changes, in particular at least 10, 12, 14, 16, 17, 18, 19, and in particular preferably 20, of these changes.

Very particularly preferred embodiments of the synthetic phytase have one of the following cumulative sums of changes with respect to SEQ ID 18:
a) A89T/D92A/H143Y/N202S/K207E/A209S/H228Y/K234I/K251N/ Q256H/H413Q
b) A89T/D92N/A142T/H143Y/N202S/K207E/A2095/H228Y/K234I/D247K/ K251N/Q256H/F356L/H413Q
c) A89T/D92A/H143Y/T156R/N202S/K207E/A209S/H228Y/K234I/ K251N/Q256H/S314G/H413Q or
d) A89T/D92A/A142T/H143Y/N202S/K207E/A209S/H228Y/K234I/D247K/ K251N/Q256H/H413Q

The respective individual or cumulative mutations, depending on position and amino acid replaced, can cause an increase in the thermal stability of the synthetic phytase by 1 to 11° C., and so a desired thermal stability of the phytase corresponding to the respective use can be selected by selecting the corresponding number and type of mutations.

These particularly preferred cumulative mutations of the synthetic phytase having nos. A-518, A-521, A-534 and A-519 (see table 1 for definitions) yield in each case an increase in the thermal stability by at least 10° C. over the synthetic phytase of SEQ ID 18 (Fus5#2). For these particularly preferred embodiments, thermostabilities result thereby of at least 10° C. above the 65° C. which has already been achieved for the phytase FusS#2 (SEQ ID 18). The temperature profile, the pH profile and the thermal stability of the phytase of SEQ ID 18 is shown respectively in FIGS. 1, 2 and 3.

In one embodiment, the synthetic phytase has at least one conservative amino acid replacement at the stated positions with respect to one of the following above-described phytases:

SIN; A6V; K12N; S17N; A84V; A89T; D92E; D92P; D92A D92N; Q109K; M137L; D138N; S140P; A142T; H143Y; Q149H; T156R; N202S; N202T; G205R; K207E; K207T; K2071; V208M; A209S; H228Y; K234N; K2341; E243K; D247K; S248L; K251N; K2511; A255V; Q256Y; Q256H; S261E; N270K; A304V; S314G; T320L; Q349R; F356L; S3731; E382G; T3991; K402N; H413L; H413Q;

### A89T/D92A/H143Y/N202S/K207E/A209S/H228Y/K234I/K251N/Q256H/H413Q; A89T/D92N/A142T/H143Y/N202S/K207E/A209S/H228Y/K2341/D247K/K251N/Q256H/ F356L/H413Q;

### A89T/D92A/H143Y/T156R/N202S/K207E/A209S/H228Y/K2341/K251N/Q256H/S314G/ H413Q and

A89T/D92A/A142T/H143Y/N202S/K207E/A209S/H228Y/K2341/D247K/ K251N/Q256H/H413Q based on SEQ ID 18,
wherein the synthetic phytase can have at least one of the individually mentioned changes or one of said groups of changes. Conservative, for the purposes of the present invention, means a replacement of the amino acid G by A; A by G, S; V by I,L,A,T,S; I by V,L,M; L by I,M,V; M by L,I,V; P by A,S,N; F by Y,W,H; Y by F,W,H; W by Y,F,H; R by K,E,D; K by R,E,D; H by Q,N,S; D by N,E,K,R,Q; E by Q,D,K,R,N; S by T,A; T by S,V,A; C by S,T,A; N by D,Q,H,S; Q by E,N,H,K,R. In this case it is possible to combine any conservative replacement of an amino acid with any conservative replacement of another amino acid.

Advantageously, the synthetic phytase is an isolated phytase. It is also conceivable that the synthetic phytase is present not as purified isolated phytase, but as fermentation broth, wherein the biomass is removed wholly, in part or not at all. In this case the broth can be concentrated or completely dried by liquid removal. It is possible to use these unpurified or partially purified phytase solutions or solids as an additive in different products.

The synthetic phytase according to the invention advantageously has an elevated thermal stability and/or an elevated specific activity with respect to the two wild type phytases from the organisms Yersinia mollaretii and Hafnia sp., which were the basis of the construction of the synthetic phytase construct according to SEQ ID 18.

The invention further comprises an isolated nucleic acid sequence encoding one of the phytases according to the invention as per the above description having the said possible changes at single positions or a plurality of positions, in particular a phytase having changes at the following amino acid positions based on SEQ ID 18:

### a) A89T/D92A/H143Y/N202S/K207E/A209S/H228Y/K2341/K251N/ Q256H/H413Q or

### b) A89T/D92N/A142T/H143Y/N202S/K207E/A2095/H228Y/K234I/ D247K/K251 N/Q256H/F356L/H413Q or

### c) A89T/D92A/H143Y/T156R/N202S/K207E/A209S/H228Y/K234I/ K251 N/Q256H/5314G/H413Q or

### d) A89T/D92A/A142T/H143Y/N202S/K207E/A209S/H228Y/K234I/ D247K/K251N/Q256H/H413Q.

The invention likewise comprises an isolated nucleic acid sequence encoding an enzyme having phytase activity, wherein the nucleic acid sequence has at least 70% identity with the nucleic acid sequence of SEQ ID 19, or a nucleic acid sequence which hybridizes with the complementary strand of one of the abovementioned sequences having at least 70% identity with the nucleic acid sequence of SEQ ID 19 under highly stringent conditions. In a particular embodiment, the isolated nucleic acid sequence has more than 70% identity, in particular 75, 80, 85, 90, 91, 92, 93, 94, 95, 96, 97, 98, 99% identity, with the SEQ ID 19.

The invention further comprises a recombinant expression vector comprising one of the nucleic acid sequences according to the invention.

The invention likewise comprises a recombinant host cell comprising one of the nucleic acid sequences according to the invention or comprising the recombinant expression vector according to the invention.

The object is additionally achieved by a recombinant production organism, wherein it is a non-human production organism which comprises one of the nucleic acid sequences according to the invention or which comprises the recombinant expression vector according to the invention. Particularly preferably, the recombinant production organism is one of the genus Aspergillus, Trichoderma, Hansenula, Saccharomyces, Escherischia, Kluyveromyces, Schizosaccharomyces.

The object is additionally achieved by an animal feed additive which comprises at least one of the phytases according to the invention and also further customary feed additives, for example for cattle, poultry or pigs, such as, for example, vitamins, minerals or other additives.

The object is additionally achieved by an animal feed which comprises at least one of the described synthetic phytases according to the invention together with customary animal feed components. In this case, all feed components are conceivable as are customarily used in feed pellets for growing cattle, dairy cows, poultry or pigs.

The invention is further achieved by using one of the described synthetic phytases according to the invention or the animal feed additive according to the invention comprising at least one of the synthetic phytases according to the invention in an animal feed. The use can be in the form of addition of the phytase according to the invention or of the animal feed additive according to the invention to the remaining feed components prior to pelleting. It is also conceivable that the phytase, after the production of feed pellets, can be applied to these pellets, in particular in liquid form.

The invention is further achieved by the use of one of the above-described synthetic phytases according to the invention, the animal feed additive according to the invention which comprises at least one of the synthetic phytases according to the invention, or the animal feed which comprises at least one of the described synthetic phytases for reducing the phosphate content in the manure of farm animals.

The described embodiments serve for explanation and for better understanding of the invention and are in no way to be taken as limiting. Further features of the invention result from the description of preferred embodiments hereinafter in combination with the subclaims. In this case the individual features of the invention can be realized in an embodiment in each case singly or in a plurality and are in no way a restriction of the invention to the described embodiment. The wording of the claims is hereby explicitly made subject matter of the description.

### DESCRIPTION OF THE FIGURES

FIG. 1 shows the temperature profile of the phytase FusS#2. The phytase activity is determined at the temperature stated in each case. For determining the relative activity values, the highest measured activity is set to 100%.

FIG. 2 shows the pH profile of the phytase FusS#2. The phytase activity is determined at the pH stated in each case. For determining the relative activity values, the highest measured activity is set to 100%.

FIG. 3 shows the temperature stability of the phytase FusS#2. The phytase is heated to the temperature stated at pH 5.5 for 20 min. After cooling, the residual activity is determined at pH 5.5 and 37° C. For determining the relative residual activity, the activity of a reference sample which is incubated at room temperature for 20 min is set to 100%.

FIG. 4 shows the plasmid map of the expression plasmid pFus5#2.

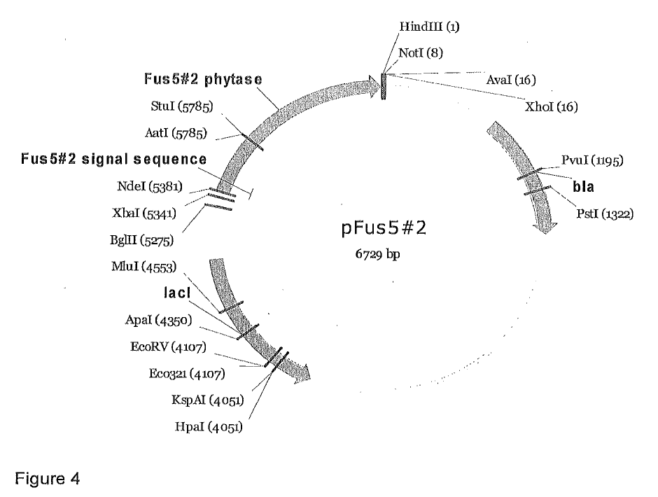

FIG. 5 shows the plasmid map of the expression plasmid pH6-Fus5#2.

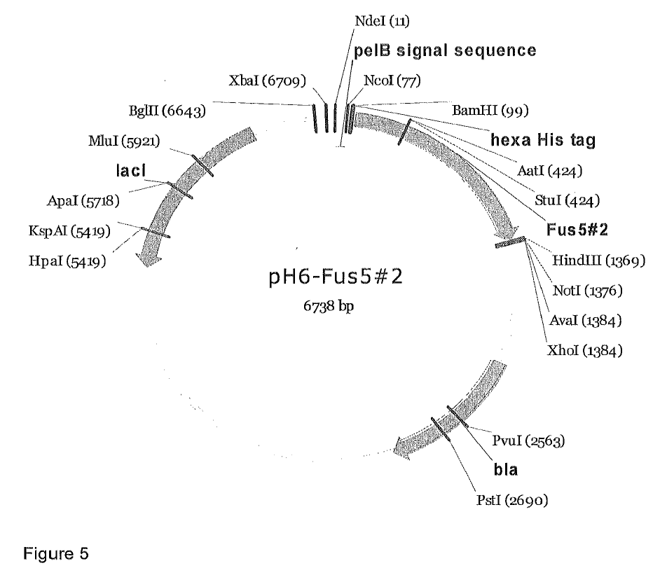

FIG. 6 shows the plasmid map of the expression plasmid pGLA53-Fus5#2.

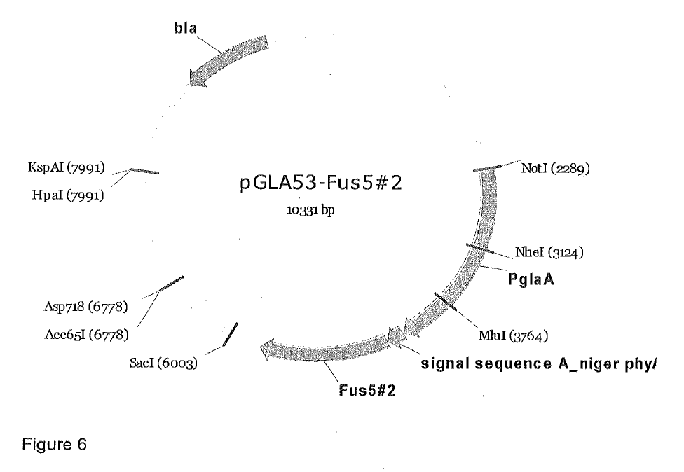

### EXAMPLES

### Cloning the Phytase from Hafnia sp. LU11047

Phytases were sought by means of PCR in a number of enterobacteria in a similar manner to the publications Huang et al. (2006) A novel phytase with preferable characteristics from Yersinia intermedia. Biochem Biophys Res Commun 350: 884-889, Shi et al. (2008) A novel phytase gene appA from Buttiauxella sp. GC21 isolated from grass carp intestine. Aquaculture 275:70-75 and WO2008116878 (example 1) using the degenerated oligos Haf1090 5′-GAYCCNYTNTTYCAYCC-3′ (SEQ ID NO: 1) and Haf1092 5′-GGNGTRTTRTCNGGYTG-3′ (SEQ ID NO: 2) at annealing temperatures between 40° C. and 50° C. The PCR products formed are used under the same annealing conditions as templates for a semi-nested PCR using the oligos Haf1090 5′-GAYCCNYTNTTYCAYCC-3′ (SEQ ID NO: 1) and Haf1091 5′-GCDATRTTNGTRTCRTG-3′ (SEQ ID NO: 3). A fragment can be isolated from a bacterial strain of the genus Hafnia (Hafnia sp. LU11047). The isolated fragment is subcloned using the “TOPO TA Cloning Kit” (Invitrogen) according to the manufacturer's instructions and subsequently sequenced. Starting from this partial sequence, the whole-length sequence of the phytase is amplified via what is termed the TAIL-PCR method (Yao-Guang Liu and Robert F. Whittier (1995) Thermal asymmetric interlaced PCR: automatable amplification and sequencing of insert end fragments from P1 and YAC clones for chromosome walking. Genomics 25, 674-681). For this purpose the following oligonucleotides are used:

Amplification of the 3′ end:

1.
Haf1165
(5′-WCAGNTGWTNGTNCTG-3′, SEQ ID NO: 4)
and
Haf1167
(5′-CTTCGAGAGCCACTTTATTACCGTCG-3′, SEQ ID NO: 5)
2.
Haf1165
(5′-WCAGNTGWTNGTNCTG-3′, SEQ ID NO: 4)
and
Haf1168
(5′-CCAATGTTGTGCTGCTGACAATAGG-3′, SEQ ID NO: 6)
3.
Haf1165
(5′-WCAGNTGWTNGTNCTG-3′, SEQ ID NO: 4)
and
Haf1169
(5′-CCGAACTCATCAGCGCTAAAGATGC-3′, SEQ ID NO: 7)

Amplification of the 5′ end:

1.
Haf1077
(5′- CAWCGWCNGASASGAA-3′, SEQ ID NO: 8)
and
Haf1170
(5′- CGCAGTTTGACTTGATGTCGCGCACG-3′, SEQ ID NO: 9)
2.
Haf1077
(5′- CAWCGWCNGASASGAA-3′, SEQ ID NO: 8)
and
Haf1171
(5′- GTCGCGCACGCCCTATATCGCCAAGC-3′, SEQ ID NO: 10)
3.
Haf1077
(5′- CAWCGWCNGASASGAA-3′, SEQ ID NO: 8)
and
Haf1172
(5′- CTGCAAACCATCGCACACGCACTGG-3′, SEQ ID NO: 11)

The DNA fragments obtained are cloned using the “TOPO TA Cloning® Kit”

(Invitrogen) and sequenced. The nucleotide sequences yield the gene SEQ ID NO: 12 encoding the phytase from Hafnia sp. LU11047. The amino acid sequence SEQ ID NO: 13 derived therefrom is 98% identical with the phytase sequence from WO200811678 of a Hafnia alvei phytase.

Using the software SignaIP 2.0, the amino acids 1-33 are predicted as a signal peptide. The mature enzyme therefore begins with the serine in position 34.

1. Synthetic Phytase Fus5#2

Cloning the Phytase Fus5#2

Starting from the chromosomal DNA of Hafnia sp. LU11047, a fragment from base 1-1074 of the phytase is amplified (SEQ ID NO: 14) by means of PCR. From the DNA sequence of a potential phytase (or acid phosphatase) from Yersinia mollaretii ATCC43969, NCBI sequence ID ZP_{—}00824387, oligonucleotides are derived for amplifying the nucleotides 1057-1323. A second phytase fragment from the chromosomal DNA of Yersinia mollaretii ATCC 43969 is amplified thereby (SEQ ID NO: 15). In the amplification of the two phytase fragments, both at the 3′ end of the Hafnia fragment and also at the 5′ end of the Yersinia fragment, with the aid of the oligos used, an overlap of 20 by with the other phytase fragment respectively is generated. In this manner, both fragments can be combined via a PCR fusion to give the phytase sequence SEQ ID NO: 16 which encodes the synthetic phytase Fus5#2. For the amino acid sequence SEQ ID NO: 17 derived therefrom, by means of the software SignalP 2.0, the amino acids 1-33 are predicted as a signal peptide. The mature phytase Fus5#2 (SEQ ID NO: 18) is encoded by the nucleotide sequence SEQ ID NO: 19.

For cloning an expression plasmid for E. coli, at the 5′ end of the phytase DNA fragment SEQ ID NO: 16 a Ndel restriction site is established, and at the 3′ end a stop codon and a HindIII restriction site is established. The sequences additionally required for this are introduced by means of a PCR reaction via the primer used, using the phytase SEQ ID NO: 16 as template. Using these sites, the gene encoding the phytase is cloned into the E. coli expression vector pET22b (Novagen). By using the Ndel restriction site and by introducing the stop codon, the pelB signal sequence is removed from the vector and a readthrough into the 6xHis tag present on the plasmid is prevented. The plasmid pFus5#2 (SEQ ID NO: 20) thus produced is transformed into the E. coli strain BL21(DE3) (Invitrogen).

For improved purifying of the phytase protein, a phytase variant having an N-terminal 6xHis tag is cloned. Using the sense-oligo primer H6: 5′-ctatggatccgcatcatcatcatcatcacagtgataccgcccctgc-3′ (SEQ ID NO: 21), which introduces both the 6xHis tag and also a BamHI site, and the sequence SEQ ID NO: 19 encoding the mature phytase protein as template, a PCR product is amplified. At the 3′ end of the PCR product, using the same antisense oligo as previously, again a stop codon and a Ndel restriction site is introduced. The fragment thus produced is cloned via BamHI/Ndel into the vector pET22b and the plasmid pH6-Fus5#2 (SEQ ID NO: 22) is obtained which is likewise transformed into E. coli BL21(DE3). In the case of this construct, the pelB signal sequence present in pET22b is used for transport into the periplasma.

Expression of the Phytase Fus5#2 in Escherichia coli

The E. coli BL21(DE3) strains which carry a plasmid having a phytase expression cassette are grown in LB medium containing ampicillin (100 mg/l) at 37° C. The phytase expression is induced at an OD (600 nm) of 0.6 by adding 1 mM IPTG. After 4 h of induction, 10% (v/v) of a 10× BugBuster solution (Novogen) is added and the mixture is incubated at room temperature for 15 min. After centrifugation, the supernatant is used for determining the phytase activity.

Purification Via Ni-Affinity Chromatography

For purifying the 6xHis-labeled phytase variants, an induced, phytase-expressing E. coli culture broth is admixed with 300 mM NaCl, Complete™ Protease Inhibitor without EDTA (according to information from the manufacturer Roche Applied Science) and admixed with 10% (v/v) of a 10× BugBuster solution (Novogen) and incubated for 15 min at room temperature. After centrifugation, the supernatant is bound to Ni-NTA columns/KIT (Qiagen) according to the manufacturer's instructions. The elution subsequently to the washing steps is performed using cold elution buffer (50 mM Na acetate buffer, 300 mM NaCl, 500 mM imidazole, 1 mM CaCl₂). Before determining the protein content, the sample buffer is exchanged by dialysis for 2 mM sodium citrate pH 5.5.

Expression of the Phytase FusS#2 in Aspergillus niger

For expressing the phytase Fus5#2 in Aspergillus niger, first an expression construct is established which contains the phytase gene under the control of the A. niger glucoamylase (glaA) promoter, flanked by the non-coding 3′ glaA region. In this manner, the construct is determined for an integration into the 3′ glaA region in A. niger. As a signal sequence for the extracellular protein secretion, the signal sequence of the A. ficuum phytase is used. As a base for the expression construct, the plasmid pGBGLA-53 (which is also called pGBTOPFYT-1 in WO9846772) which is described in detail in EP0635574B1 is used. Using the PCR-based cloning methods known to those skilled in the art, in pGBGLA-53 the gene section of the A. ficuum phytase, which encodes the mature phytase protein starting with the amino acid sequence ASRNQSS, is replaced by the gene section SEQ ID NO: 19 encoding the mature Fus5#2 phytase. The resultant plasmid pGLA53-FusS#2 (SEQ ID NO: 23) is formed. The co-transformation of the linear expression cassette isolated from the resultant plasmid by HindII together with an amdS marker cassette isolated from the plasmid pGBLA50 (EP0635574B1)/pGBAAS-1 (name of the same plasmid in WO9846772) into a glaA-deleted A. niger expression strain and the resultant expression of the phytase in shake flasks proceeds as described in the two patent documents cited. The phytase activity in the culture supernatant is determined daily after centrifuging off the cells. The maximum activity is achieved between day 3 and day 6.

Determination of the Specific Activity

The phytase activity is determined in microtiter plates. The purified enzyme sample is diluted in reaction buffer (250 mM Na acetate, 1 mM CaCl₂, 0.01% Tween 20, pH 5.5). 10 μl of the enzyme solution are incubated at 60° C. for 20 min with 110 μl of substrate solution (6 mM Na phytate (Sigma P3168) in reaction buffer). The reaction is stopped by adding 80 μl of trichloroacetic acid solution (15% w/w). 20 μl of the stopped reaction solution, for detecting the phosphate released, are admixed with 280 μl of freshly made up staining reagent (60 mM L-ascorbic acid (Sigma A7506), 2.2 mM ammonium molybdate tetrahydrate, 325 mM H₂SO₄) and incubated at 50° C. for 25 min and subsequently the absorption is determined at 820 nm. As a blank value the substrate buffer is incubated alone at 37° C. and 10 μl of enzyme sample is added only after stopping with trichloroacetic acid. The staining reaction is performed in a similar manner. The amount of phosphate released is determined via a calibration curve of the staining reaction using a phosphate solution of known concentration. The enzyme activity which releases 1 μmol of phosphate per min under these conditions is termed 1 U. The protein concentration of the phytase solution used is determined from the absorption at 280 nm. For this purpose, the molecular extinction coefficient of the phytase is determined using the “Vector NTI” software (Invitrogen, version 10.3.0). The specific activity of the Fus5#2 phytase is 2300+/−200 U/mg.

Phytase Assay

The phytase activity is determined in microtiter plates. The enzyme sample is diluted in reaction buffer (250 mM Na acetate, 1 mM CaCl₂, 0.01% Tween 20, pH 5.5). 10 μl of the enzyme solution are incubated at 37° C. for 1 h with 140 μl of substrate solution (6 mM Na phytate (Sigma P3168) in reaction buffer). The reaction is stopped by adding 150 μl of trichloroacetic acid solution (15% w/w). 20 μl of the stopped reaction solution, for detecting the phosphate released, are admixed with 280 μl of freshly made up staining reagent (60 mM L-ascorbic acid (Sigma A7506), 2.2 mM ammonium molybdate tetrahydrate, 325 mM H₂SO₄) and incubated at 50° C. for 25 min and then the absorption is determined at 820 nm. As a blank value, the substrate buffer is incubated alone at 37° C. and the 10 μl enzyme sample is added only after stopping with the trichloroacetic acid. The staining reaction proceeds in a similar manner to the remaining measured values. The amount of phosphate released is determined via a calibration curve of the staining reaction using a phosphate solution of known concentration.

Determination of the Temperature Optimum

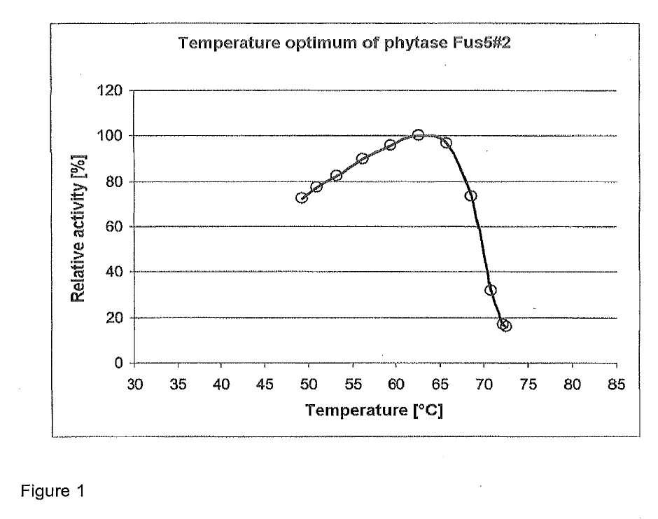

For determining the temperature optimum, the enzyme sample is diluted in reaction buffer (250 mM Na acetate, 1 mM CaCl₂, 0.01% Tween 20, pH 5.5). 10 μl of the pre-tempered (5 min, respective reaction temperature) enzyme solution are incubated for 30 min with 110 μl of pretempered substrate solution (6 mM Na phytate (Sigma P3168) in reaction buffer). The incubation proceeds at various temperatures in a gradient heating block. The reaction is stopped by adding 80 μl of trichloroacetic acid solution (15% w/w). 20 μl of the stopped reaction solution, for detecting the phosphate released, are admixed with 280 μl of freshly made up staining reagent (60 mM L-ascorbic acid (Sigma A7506), 2.2 mM ammonium molybdate tetrahydrate, 325 mM H₂SO₄) and incubated at 50° C. for 25 min and then the absorption is determined at 820 nm. As a blank value, the substrate buffer alone is incubated at the stated temperature and 10 μl of enzyme sample are added, only after stopping with trichloroacetic acid. The staining reaction proceeds in a similar manner to the other measured values. For determining the relative activity, the highest measured activity is set to 100%. The results are shown in FIG. 1.

Temperature profile of the phytase Fus5#2:

| | Temperature [° C.] | | | | | | | | | | |
|---|---|---|---|---|---|---|---|---|---|---|---|
| | 49.4 | 51 | 53.3 | 56.2 | 59.4 | 62.6 | 65.8 | 68.5 | 70.8 | 72.2 | 72.6 |
| Relative | 72.3 | 77.4 | 82.2 | 89.6 | 95.5 | 100.0 | 96.5 | 73.1 | 31.7 | 16.9 | 15.9 |
| activity [%] | | | | | | | | | | | |

The temperature optimum of the Fus5#2 phytase is approximately 63° C.

Determination of the pH Optimum

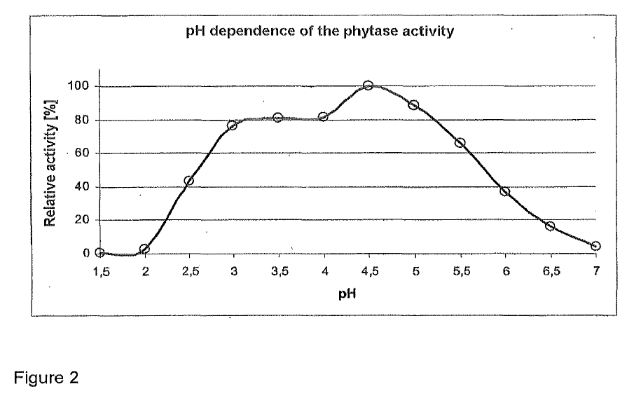

For determining the pH optimum, a modified reaction buffer (100 mM Na acetate, 100 mM glycine, 100 mM imidazole, 1 mM CaCl₂, 0.01% Tween 20) is used for the phytase assay, which modified reaction buffer is adjusted using dilute hydrochloric acid to pHs in the range pH 1.5-7. For determining the relative activity, the highest measured activity is set to 100%. The results are shown in FIG. 2.

pH profile of the phytase Fus5#2:

| | pH | | | | | | | | | | | |
|---|---|---|---|---|---|---|---|---|---|---|---|---|
| | 1.5 | 2 | 2.5 | 3 | 3.5 | 4 | 4.5 | 5 | 5.5 | 6 | 6.5 | 7 |
| Relative | 0.8 | 2.8 | 43.2 | 76.1 | 81.1 | 81.5 | 100.0 | 88.9 | 66.1 | 37.2 | 15.9 | 4.1 |
| activity [%] | | | | | | | | | | | | |

The pH optimum of the Fus5#2 phytase is pH 4.5.

Determination of the Thermal Stability (T₅₀)

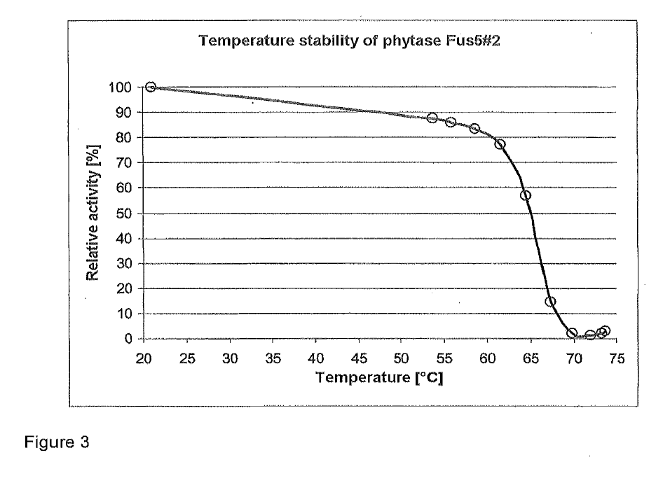

For recording the thermal inactivation curve, the diluted enzyme sample in reaction buffer (250 mM Na acetate, 1 mM CaCl₂, 0.01% Tween 20, pH 5.5) is heated to the respective temperatures for 20 min and then cooled to 4° C. A non-thermally treated reference sample is left at room temperature for 20 min and thereafter likewise cooled to 4° C. Subsequently to the thermal pretreatment, the enzyme activity of the samples is determined by means of the phytase assay. The activity of the reference sample is normalized to 100%. The thermal stability of the various phytase variants is characterized by the T₅₀ value. The T₅₀ value gives the temperature at which, after thermal inactivation, 50% of residual activity still exists compared with a reference sample which is not thermally treated. Changes in the thermal stability of two phytase variants, expressed in ° C., result owing to the difference of the respective T₅₀ values. The results are shown in FIG. 3.

T₅₀ determination of the phytase Fus5#2:

| | Temperature [° C.] | | | | | | | | | | |
|---|---|---|---|---|---|---|---|---|---|---|---|
| | 21 | 53.8 | 55.9 | 58.6 | 61.5 | 64.5 | 67.4 | 69.9 | 72 | 73.3 | 73.7 |
| Remaining | 100 | 88 | 86 | 83 | 77 | 57 | 15 | 2 | 1 | 2 | 3 |
| activity [%] | | | | | | | | | | | |

A T₅₀ value of 65° C. results therefrom for the Fus5#2 phytase.

2. Phytase Variants of Phytase Fus5#2

Variants of the phytase were generated by mutation of the gene sesquence SEQ ID NO: 19 by means of PCR. For a targeted mutagenesis, the “Quickchange Site-directed Mutagenesis Kit” (Stratagene) is used. A random mutagenesis over all, or else only a part, of the coding sequence of SEQ ID NO: 19 is carried out using the “GeneMorph II Random Mutagenesis Kit” (Stratagene). The mutagenesis rate is set to the desired degree of 1-5 mutations via the amount of template DNA used. Multiple mutations are generated by the targeted combination of single mutations or by sequentially carrying out a plurality of mutagenesis rounds.

Variants of the phytase gene generated in this manner are cloned in a similar manner to the original phytase Fus5#2 into the E. coil expression vector pET22b (Novagen) and then expressed using the E. coli strain BL21(DE3).

In a few cases, selected phytase variants are expressed in a similar manner to the starting phytase Fus5#2 using a corresponding expression construct in Aspergillus niger.

The phytase variants generated are tested for phytase activity and temperature stability in a high-throughput test. For this purpose, the E. coli BL21(DE3) clones obtained after transformation with the pET22b-based expression construct are incubated in 96-well microtiter plates in LB medium (2% glucose, 100 mg/l ampicillin) (30° C., 900 rpm, 2 mm shaker deflection). At an OD (600 nm) of approximately 0.5, induction is performed using 1 mM IPTG for 4 h. Subsequently, 10% (v/v) of a 10× BugBuster solution (Novogen) is added and the mixture is incubated for 15 min at room temperature. Phytase activity and residual activity after a 20 minute temperature stress are determined. For variants having an elevated relative residual activity, the thermal stability (T₅₀ ) is determined. For some selected phytase variants, additional characteristic parameters (e.g. temperature optimum, specific activity, pH optimum) are determined.

Thermal Stability

The increase in the thermal stability of the individual phytase variants is expressed by ΔT, wherein ΔT gives the increase in ° C. of the T₅₀ value compared with the phytase Fus5#2. The mutation details relate to the starting molecule Fus5#2.

TABLE 1
Increase in the thermal stability of the phytase
variants having 1 to 14 mutations compared with
the synthetic phytase Fus5#2 in ° C.

| Mutant | | ΔT |
|---|---|---|
| numbers | Mutation | [° C.] |
| Fus5#2 | SEQ ID NO 18 | 0 |
| A-4 | Q349R | 1 |
| A-10 | A84V/A304V | 1 |
| A-66 | H228Y | 1 |
| A-73 | N202S | 1 |
| C-7 | T320L/H413L | 1 |
| C-40 | K234N | 1 |
| X-1 | Q256Y | 1 |
| X-2 | K207E/A209S/N270K | 1 |
| A-164 | A6V | 1 |
| B-16 | K207E | 1 |
| B-378 | H143Y | 1 |
| C-79 | Q109K/D247K | 1 |
| A-11 | Q256H/K402N | 1.5 |
| X-6 | K207E/A209S | 1.5 |
| B-320 | M137L/K207T | 1.5 |
| A-508 | Q349R/H228Y/A304V | 1.5 |
| A-8 | K234I/K251N/H413Q | 2 |
| A-20 | D92E | 2 |
| A-507 | N202S/H228Y | 2 |
| X-3 | D92P | 2.5 |
| A-505 | D92E/N202S | 2.5 |
| A-501 | D92E/K234I/K251N/H413Q | 3 |
| A-407 | A89T/D92A/N270K | 3 |
| A-502 | D92E/Q256H | 3.5 |
| X-4 | A89T/D92A | 3 |
| A-408 | A89T/D92A/K207E/A209S | 3.5 |
| A-415 | A89T/D92A/S261E | 3.5 |
| A-501 | D92E/K234I/K251N/H413Q | 3.5 |
| A-409 | A89T/D92A/S248L/Q256Y | 4 |
| A-503 | D92E/K234I/K251N/Q256H/H413Q | 5 |
| A-406 | A89T/D92A/Q256Y | 5 |
| A-510 | D92E/N202S/K234I/K251N/Q256Y/H413Q/ | 5 |
| | K207E/A209S | |
| A-515 | D92E/N202S/A209S/K234I/Q256Y/H413Q | 5 |
| D-5 | D92E/A142T/K234I/K251N/Q256H/H413Q | 5.5 |
| D-34 | S1N/S17N/D92E/K234I/K251N/Q256H/H413Q | 5.5 |
| F-161 | K12N/D92E/K234I/K251N/Q256H/H413Q | 5.5 |
| A-504 | D92E/N202S/K234I/K251N/Q256H/H413Q | 6 |
| D-192 | D92E/S140P/K207I/K234I/K251N/Q256H/ | 6 |
| | H413Q | |
| A-511 | D92E/M137L/N202S/K234I/K251N/Q256H/ | 6 |
| | H413Q | |
| A-514 | D92E/N202S/K234I/K251N/Q256H/K402N | 6 |
| | H413Q | |
| A-516 | D92E/N202S/K234I/E243K/K251N/Q256H/ | 6 |
| | H413Q | |
| F-41 | D92E/D138N/K234I/K251N/Q256H/H413Q | 6.5 |
| D-207 | D92E/Q149H/K234I/K251N/Q256H/H413Q | 6.5 |
| D-268 | D92E/T156R/K234I/K251N/Q256H/H413Q | 6.5 |
| F-150 | D92E/K234I/K251N/A255V/Q256H/H413Q | 6.5 |
| I-117 | D92E/N202T/K234I/K251N/Q256H/S373I/ | 6.5 |
| | E382G/T399I/H413Q | |
| A-509 | A89T/D92A/N202S/K234I/K251N/Q256H/ | 6.5 |
| | H413Q | |
| H-107 | D92E/N202S/K234I/K251N/Q256H/H413Q | 7 |
| H-159 | A89T/D92A/N202S/K207E/K234I/K251N/ | 7 |
| | Q256H | |
| H-456 | A89T/D92A/K207E/K234I/K251N/Q256H/ | 7 |
| | H413Q | |
| A-512 | D92E/H143Y/K234I/K251N/Q256H/H413Q | 7 |
| H-464 | A89T/D92A/G205R/K207E/V208M/K251N/ | 7.5 |
| | Q256H | |
| A-513 | D92E/H/228Y/K234I/K251N/Q256H/H413Q | 7.5 |
| A-518 | A89T/D92A/H143Y/N202S/K207E/A209S/ | 10 |
| | H228Y/K234I/K251N/Q256H/H413Q | |
| A-521 | A89T/D92N/A142T/H143Y/N202S/K207E/ | 10 |
| | A209S/H228Y/K234I/D247K/K251N/Q256H/ | |
| | F356L/H413Q | |
| A-534 | A89T/D92A/H143Y/T156R/N202S/K207E/ | 11 |
| | A209S/H228Y/K234I/K251N/Q256H/S314G/ | |
| | H413Q | |
| A-519 | A89T/D92A/A142T/H143Y/N202S/K207E/ | 11 |
| | A209S/H228Y/K234I/D247K/K251N/Q256H/ | |
| | H413Q | |

TABLE 2
Increase in specific activity of phytase variants
compared with the synthetic phytase Fus5#2.

| | | Relative specific |
|---|---|---|
| Mutant numbers | Mutation | activity [%] |
| Fus5#2 | SEQ ID NO 18 | 100 |
| A-8 | K234I/K251I/ | 111 |
| | H413Q | |
| X-5 | D92N | 120 |

1-19. (canceled)

20. A phytase which has at least 90% identity with the amino acid sequence of SEQ ID 18.

21. The phytase according to claim 20, wherein it has a change of the amino acid at at least one of the positions selected from the group consisting of position 1, 6, 12, 17, 84, 89, 92, 109, 137, 138, 140, 142, 143, 149, 156, 202, 205, 207, 208, 209, 228, 234, 243, 247, 248, 251, 255, 256, 261, 270, 304, 314, 320, 349, 356, 373, 382, 399, 402 and 413, based on the position according to SEQ ID 18.

22. The phytase according to claim 21, wherein it has at least 5 of the changes.

23. The phytase according to claim 20, wherein at least one of the amino acids at position 1, 6, 12, 17, 84, 89, 92, 109, 137, 138, 140, 142, 143, 149, 156, 202, 205, 207, 208, 209, 228, 234, 243, 247, 248, 251, 255, 256, 261, 270, 304, 314, 320, 349, 356, 373, 382, 399, 402 and 413, based on the position according to SEQ ID 18, is replaced by in each case one of the amino acids 1 N, D, Q, H; 6 V, I, L, T, S; 12 N, D, Q, H, S; 17 N, D, Q, H; 84 V, I, L, T, S; 89 T, S, V; 92 E, Q, K, R, N, P, A, S, G, H; 109 K, R, E, D; 137 L, I, V; 138 N, Q, H, S; 140 P, A, N; 142 T, S, V; 143 Y, F, W; 149 H, N, S; 156 R, K, E, D; 202 S, T, A, V; 205 R, K, E, D; 207 E, Q, D, R, N, T, S, V, A, I, L, M; 208 M, L, I; 209 S, T; 228 Y, F, W; 234 N, D, Q, H, S, I, V, L, M; 243 K, R, D; 247 K, R, E; 248 L, I, M, V; 251 N, D, Q, H, S, I, V, L, M; 255 V, I, L, T, S; 256 Y, F, W, H, N, S; 261 E, Q, D, K, R, N; 270 K, R, E, D; 304 V, I, L, T, S; 314 G, A; 320 L, I, M, V; 349 R, K, E, D; 356 L, I, M, V; 373 I, V, L, M; 382 G, A; 399 I, V, L, M; 402 N, D, Q, H, S; 413 L, I, M, V, Q, E, N, K, R.

24. The phytase according to claim 20, wherein it has at least one of the changes selected from the group consisting of SIN; A6V; K12N; S17N; A84V; A89T; D92E; D92P; D92A; D92N; Q109K; M137L; D138N; S140P; A142T; H143Y; Q149H; T156R; N202S; N202T; G205R; K207E; K207T; K207I; V208M; A209S; H228Y; K234N; K234I; E243K; D247K; S248L; K251N; K251I; A255V; Q256Y; Q256H; S261E; N270K; A304V; 5314G; T320L; Q349R; F356L; S373I; E382G; T399I; K402N; H413L and H413Q with respect to the amino acid sequence of SEQ ID 18.

25. The phytase according to claim 24, wherein it has at least 5 of the changes.

26. The phytase according to claim 20, wherein it has at least one of the changes selected from the group consisting of
[A89T/D92A/H143Y/N202S/K207E/A209S/H228Y/K2341/K251N/Q256H/H413Q],
[A89T/D92N/A142T/H143Y/N202S/K207E/A209S/H228Y/K234I/D247K/K251N/Q256H/F356L/H413Q],
[A89T/D92A/H143Y/T156R/N2025/K207E/A209S/H228Y/K234I/K251N/Q256H/S314G/H413Q] and
[A89T/D92A/A142T/H143Y/N202S/K207E/A209S/H228Y/K234I/D247K/K251N/Q256H/H413Q].

27. A phytase according to claim 20, wherein it has at least one conservative amino acid replacement at at least one position with respect to one of the phytases according to claim 24.

28. The phytase according to claim 20, wherein it is an isolated phytase.

29. The phytase according to claim 20, wherein it has an elevated thermal stability and/or an elevated specific activity with respect to the two wild type phytases from Yersinia mollaretii and Hafnia sp.

30. An isolated nucleic acid sequence encoding a phytase which encodes one of the phytases according to claim 20.

31. An isolated nucleic acid sequence encoding a phytase which
a) has at least 90% identity with the nucleic acid sequence of SEQ ID 19, or
b) hybridizes with the complementary strand of one of the sequences of a) under highly stringent conditions.

32. A recombinant expression vector comprising a nucleic acid sequence according to claim 30.

33. A recombinant host cell comprising a nucleic acid sequence according to claim 30 or the vector according to claim 32.

34. A recombinant production organism comprising a nucleic acid sequence according to claim 30 or the vector according to claim 32.

35. An animal feed additive comprising at least one of the phytases according to claim 20 and also further feed additives.

36. An animal feed comprising at least one of the phytases according to claim 20.

37. The use of a phytase according to claim 20 or the animal feed additive according to claim 16 in an animal feed.

38. The use of a phytase according to claim 20, the animal feed additive according to claim 35, or the animal feed according to claim 36 for reducing the phosphate content in the manure of farm animals.


SEQUENCE LISTING (23 sequences)

SEQ ID NO: 1 - DNA, 17 bp, Artificial Sequence
  Primer
  misc_feature (6)..(6): n is a, c, g, or t
  misc_feature (9)..(9): n is a, c, g, or t
gayccnytnttycaycc

SEQ ID NO: 2 - DNA, 17 bp, Artificial Sequence
  Primer
  misc_feature (3)..(3): n is a, c, g, or t
  misc_feature (12)..(12): n is a, c, g, or t
ggngtrttrtcnggytg

SEQ ID NO: 3 - DNA, 17 bp, Artificial Sequence
  Primer
  misc_feature (9)..(9): n is a, c, g, or t
gcdatrttngtrtcrtg

SEQ ID NO: 4 - DNA, 16 bp, Artificial Sequence
  Primer
  misc_feature (5)..(5): n is a, c, g, or t
  misc_feature (10)..(10): n is a, c, g, or t
  misc_feature (13)..(13): n is a, c, g, or t
wcagntgwtngtnctg

SEQ ID NO: 5 - DNA, 26 bp, Artificial Sequence
  Primer
cttcgagagccactttattaccgtcg

SEQ ID NO: 6 - DNA, 25 bp, Artificial Sequence
  Primer
ccaatgttgtgctgctgacaatagg

SEQ ID NO: 7 - DNA, 25 bp, Artificial Sequence
  Primer
ccgaactcatcagcgctaaagatgc

SEQ ID NO: 8 - DNA, 16 bp, Artificial Sequence
  Primer
  misc_feature (8)..(8): n is a, c, g, or t
cawcgwcngasasgaa

SEQ ID NO: 9 - DNA, 26 bp, Artificial Sequence
  Primer
cgcagtttgacttgatgtcgcgcacg

SEQ ID NO: 10 - DNA, 26 bp, Artificial Sequence
  Primer
gtcgcgcacgccctatatcgccaagc

SEQ ID NO: 11 - DNA, 25 bp, Artificial Sequence
  Primer
ctgcaaaccatcgcacacgcactgg

SEQ ID NO: 12 - DNA, 1341 bp, Hafnia sp.
  sig_peptide (1)..(99)
atgacaatctctctgtttaaccgtaataaacccgctattgcacagcgtattttatgtcctctgatcgtggctttattctcaggtttaccggcatacgccagtgataccgcccctgctgggttccagttggaaaaggttgttatcctaagcagacatggcgtacgcgcgccaaccaaaatgacacaaacgatgcgcaacgtcacacctcaccagtggcctgaatggccggtaaaactcggctatatcacgccccgcggtgaacatctgattagcctgatgggcggtttttatcgagagcgctttcagcaacaaggcttattacctaaggataactgtcctacaccagatgccgtgtatgtttgggcagacgtcgatcaacgcacacgtaaaaccggcgaggccttcttagcgggtcttgctccccagtgtgatttagcgatccaccatcagcaaaacattcagcaggccgatccgctgttccatcctgtgaaagccggtatctgttcgatggataaatcacaggcacacgccgccgttgaaaagcaggcaggcacaccgattgagacgctcaatcaacgctatcaagcatctttagcgctgatgagttcggtactcgattttccaaaatccccctattgtcagcagcacaacattggcaaactctgcgatttttcacaggcgatgcctagcaggctggcgataaatgacgacggtaataaagtggctctcgaaggtgccgtgggactttcatcgacgttggctgaaattttcctgctggaacacgctcagggaatgcctaaagtggcttgggggaatattcacactgagcagcaatgggactctctgttaaaattgcataatgcgcagtttgacttgatgtcgcgcacgccctatatcgccaagcataacggtactccactgctgcaaaccatcgcacacgcactgggttccaatatcgcgagtcgcccactgccggatatttcgccagacaataagatcctgtttattgccggtcacgacaccaatattgccaatatttctggcatgctagggatgacatggacacttccgggacagccagataacacgcctccgggcggggctttagtgtttgaacgttgggtagataacgcggggaaaccgtatgttagcgtgaatatggtgtatcaaacactggcacagttgcacgaccagacgccgctaacgttgcagcatcctgcgggcagcgtacgactaaacataccgggttgcagcgatcaaacgcccgatggctattgcccgctctccaccttcagccgtttagtcaaccacagcgttgagcctgcgtgccagcttccttaa

SEQ ID NO: 13 - protein, 446 aa, Hafnia sp.
  SIGNAL (1)..(33)
MTISLFNRNKPAIAQRILCPLIVALFSGLPAYASDTAPAGFQLEKVVILSRHGVRAPTKMTQTMRNVTPHQWPEWPVKLGYITPRGEHLISLMGGFYRERFQQQGLLPKDNCPTPDAVYVWADVDQRTRKTGEAFLAGLAPQCDLAIHHQQNIQQADPLFHPVKAGICSMDKSQAHAAVEKQAGTPIETLNQRYQASLALMSSVLDFPKSPYCQQHNIGKLCDFSQAMPSRLAINDDGNKVALEGAVGLSSTLAEIFLLEHAQGMPKVAWGNIHTEQQWDSLLKLHNAQFDLMSRTPYIAKHNGTPLLQTIAHALGSNIASRPLPDISPDNKILFIAGHDTNIANISGMLGMTWTLPGQPDNTPPGGALVFERWVDNAGKPYVSVNMVYQTLAQLHDQTPLTLQHPAGSVRLNIPGCSDQTPDGYCPLSTFSRLVNHSVEPACQLP

SEQ ID NO: 14 - DNA, 1074 bp, Hafnia sp.
atgacaatctctctgtttaaccgtaataaacccgctattgcacagcgtattttatgtcctctgatcgtggctttattctcaggtttaccggcatacgccagtgataccgcccctgctgggttccagttggaaaaggttgttatcctaagcagacatggcgtacgcgcgccaaccaaaatgacacaaacgatgcgcaacgtcacacctcaccagtggcctgaatggccggtaaaactcggctatatcacgccccgcggtgaacatctgattagcctgatgggcggtttttatcgagagcgctttcagcaacaaggcttattacctaaggataactgtcctacaccagatgccgtgtatgtttgggcagacgtcgatcaacgcacacgtaaaaccggcgaggccttcttagcgggtcttgctccccagtgtgatttagcgatccaccatcagcaaaacattcagcaggccgatccgctgttccatcctgtgaaagccggtatctgttcgatggataaatcacaggcacacgccgccgttgaaaagcaggcaggcacaccgattgagacgctcaatcaacgctatcaagcatctttagcgctgatgagttcggtactcgattttccaaaatccccctattgtcagcagcacaacattggcaaactctgcgatttttcacaggcgatgcctagcaggctggcgataaatgacgacggtaataaagtggctctcgaaggtgccgtgggactttcatcgacgttggctgaaattttcctgctggaacacgctcagggaatgcctaaagtggcttgggggaatattcacactgagcagcaatgggactctctgttaaaattgcataatgcgcagtttgacttgatgtcgcgcacgccctatatcgccaagcataacggtactccactgctgcaaaccatcgcacacgcactgggttccaatatcgcgagtcgcccactgccggatatttcgccagacaataagatcctgtttattgccggtcacgacaccaatattgccaatatttctggcatgctagggatgacatggacacttccggga

SEQ ID NO: 15 - DNA, 270 bp, Yersinia mollaretii
cagcccgataacaccccgccgggtggggggctggtgtttgaactatggcagaatccagataaccatcagcaatatgtcgcagttaagatgttctatcaaacaatggatcagttacgaaatagtgaaaagttagacctgaaaagtcatccagccggtattgttcccattgagatcgaaggttgtgagaacatcggtacagacaaactttgccagcttgataccttccaaaagagagtggctcaggtgattgaacctgcatgccatatttaa

SEQ ID NO: 16 - DNA, 1344 bp, Artificial Sequence
  synthetic construct
  sig_peptide (1)..(99)
atgacaatctctctgtttaaccgtaataaacccgctattgcacagcgtattttatgtcctctgatcgtggctttattctcaggtttaccggcatacgccagtgataccgcccctgctgggttccagttggaaaaggttgttatcctaagcagacatggcgtacgcgcgccaaccaaaatgacacaaacgatgcgcaacgtcacacctcaccagtggcctgaatggccggtaaaactcggctatatcacgccccgcggtgaacatctgattagcctgatgggcggtttttatcgagagcgctttcagcaacaaggcttattacctaaggataactgtcctacaccagatgccgtgtatgtttgggcagacgtcgatcaacgcacacgtaaaaccggcgaggccttcttagcgggtcttgctccccagtgtgatttagcgatccaccatcagcaaaacattcagcaggccgatccgctgttccatcctgtgaaagccggtatctgttcgatggataaatcacaggcacacgccgccgttgaaaagcaggcaggcacaccgattgagacgctcaatcaacgctatcaagcatctttagcgctgatgagttcggtactcgattttccaaaatccccctattgtcagcagcacaacattggcaaactctgcgatttttcacaggcgatgcctagcaggctggcgataaatgacgacggtaataaagtggctctcgaaggtgccgtgggactttcatcgacgttggctgaaattttcctgctggaacacgctcagggaatgcctaaagtggcttgggggaatattcacactgagcagcaatgggactctctgttaaaattgcataatgcgcagtttgacttgatgtcgcgcacgccctatatcgccaagcataacggtactccactgctgcaaaccatcgcacacgcactgggttccaatatcgcgagtcgcccactgccggatatttcgccagacaataagatcctgtttattgccggtcacgacaccaatattgccaatatttctggcatgctagggatgacatggacacttccgggacagcccgataacaccccgccgggtggggggctggtgtttgaactatggcagaatccagataaccatcagcaatatgtcgcagttaagatgttctatcaaacaatggatcagttacgaaatagtgaaaagttagacctgaaaagtcatccagccggtattgttcccattgagatcgaaggttgtgagaacatcggtacagacaaactttgccagcttgataccttccaaaagagagtggctcaggtgattgaacctgcatgccatatttaa

SEQ ID NO: 17 - protein, 447 aa, Artificial Sequence
  synthetic construct
  SIGNAL (1)..(33)
MTISLFNRNKPAIAQRILCPLIVALFSGLPAYASDTAPAGFQLEKVVILSRHGVRAPTKMTQTMRNVTPHQWPEWPVKLGYITPRGEHLISLMGGFYRERFQQQGLLPKDNCPTPDAVYVWADVDQRTRKTGEAFLAGLAPQCDLAIHHQQNIQQADPLFHPVKAGICSMDKSQAHAAVEKQAGTPIETLNQRYQASLALMSSVLDFPKSPYCQQHNIGKLCDFSQAMPSRLAINDDGNKVALEGAVGLSSTLAEIFLLEHAQGMPKVAWGNIHTEQQWDSLLKLHNAQFDLMSRTPYIAKHNGTPLLQTIAHALGSNIASRPLPDISPDNKILFIAGHDTNIANISGMLGMTWTLPGQPDNTPPGGGLVFELWQNPDNHQQYVAVKMFYQTMDQLRNSEKLDLKSHPAGIVPIEIEGCENIGTDKLCQLDTFQKRVAQVIEPACHI

SEQ ID NO: 18 - protein, 414 aa, Artificial Sequence
  chimeric phytase
SDTAPAGFQLEKVVILSRHGVRAPTKMTQTMRNVTPHQWPEWPVKLGYITPRGEHLISLMGGFYRERFQQQGLLPKDNCPTPDAVYVWADVDQRTRKTGEAFLAGLAPQCDLAIHHQQNIQQADPLFHPVKAGICSMDKSQAHAAVEKQAGTPIETLNQRYQASLALMSSVLDFPKSPYCQQHNIGKLCDFSQAMPSRLAINDDGNKVALEGAVGLSSTLAEIFLLEHAQGMPKVAWGNIHTEQQWDSLLKLHNAQFDLMSRTPYIAKHNGTPLLQTIAHALGSNIASRPLPDISPDNKILFIAGHDTNIANISGMLGMTWTLPGQPDNTPPGGGLVFELWQNPDNHQQYVAVKMFYQTMDQLRNSEKLDLKSHPAGIVPIEIEGCENIGTDKLCQLDTFQKRVAQVIEPACHI

SEQ ID NO: 19 - DNA, 1245 bp, Artificial Sequence
  synthetic construct
agtgataccgcccctgctgggttccagttggaaaaggttgttatcctaagcagacatggcgtacgcgcgccaaccaaaatgacacaaacgatgcgcaacgtcacacctcaccagtggcctgaatggccggtaaaactcggctatatcacgccccgcggtgaacatctgattagcctgatgggcggtttttatcgagagcgctttcagcaacaaggcttattacctaaggataactgtcctacaccagatgccgtgtatgtttgggcagacgtcgatcaacgcacacgtaaaaccggcgaggccttcttagcgggtcttgctccccagtgtgatttagcgatccaccatcagcaaaacattcagcaggccgatccgctgttccatcctgtgaaagccggtatctgttcgatggataaatcacaggcacacgccgccgttgaaaagcaggcaggcacaccgattgagacgctcaatcaacgctatcaagcatctttagcgctgatgagttcggtactcgattttccaaaatccccctattgtcagcagcacaacattggcaaactctgcgatttttcacaggcgatgcctagcaggctggcgataaatgacgacggtaataaagtggctctcgaaggtgccgtgggactttcatcgacgttggctgaaattttcctgctggaacacgctcagggaatgcctaaagtggcttgggggaatattcacactgagcagcaatgggactctctgttaaaattgcataatgcgcagtttgacttgatgtcgcgcacgccctatatcgccaagcataacggtactccactgctgcaaaccatcgcacacgcactgggttccaatatcgcgagtcgcccactgccggatatttcgccagacaataagatcctgtttattgccggtcacgacaccaatattgccaatatttctggcatgctagggatgacatggacacttccgggacagcccgataacaccccgccgggtggggggctggtgtttgaactatggcagaatccagataaccatcagcaatatgtcgcagttaagatgttctatcaaacaatggatcagttacgaaatagtgaaaagttagacctgaaaagtcatccagccggtattgttcccattgagatcgaaggttgtgagaacatcggtacagacaaactttgccagcttgataccttccaaaagagagtggctcaggtgattgaacctgcatgccatatttaa

SEQ ID NO: 20 - DNA, 6729 bp, Artificial Sequence
  synthetic construct
agcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgatgacaatctctctgtttaaccgtaataaacccgctattgcacagcgtattttatgtcctctgatcgtggctttattctcaggtttaccggcatacgccagtgataccgcccctgctgggttccagttggaaaaggttgttatcctaagcagacatggcgtacgcgcgccaaccaaaatgacacaaacgatgcgcaacgtcacacctcaccagtggcctgaatggccggtaaaactcggctatatcacgccccgcggtgaacatctgattagcctgatgggcggtttttatcgagagcgctttcagcaacaaggcttattacctaaggataactgtcctacaccagatgccgtgtatgtttgggcagacgtcgatcaacgcacacgtaaaaccggcgaggccttcttagcgggtcttgctccccagtgtgatttagcgatccaccatcagcaaaacattcagcaggccgatccgctgttccatcctgtgaaagccggtatctgttcgatggataaatcacaggcacacgccgccgttgaaaagcaggcaggcacaccgattgagacgctcaatcaacgctatcaagcatctttagcgctgatgagttcggtactcgattttccaaaatccccctattgtcagcagcacaacattggcaaactctgcgatttttcacaggcgatgcctagcaggctggcgataaatgacgacggtaataaagtggctctcgaaggtgccgtgggactttcatcgacgttggctgaaattttcctgctggaacacgctcagggaatgcctaaagtggcttgggggaatattcacactgagcagcaatgggactctctgttaaaattgcataatgcgcagtttgacttgatgtcgcgcacgccctatatcgccaagcataacggtactccactgctgcaaaccatcgcacacgcactgggttccaatatcgcgagtcgcccactgccggatatttcgccagacaataagatcctgtttattgccggtcacgacaccaatattgccaatatttctggcatgctagggatgacatggacacttccgggacagcccgataacaccccgccgggtggggggctggtgtttgaactatggcagaatccagataaccatcagcaatatgtcgcagttaagatgttctatcaaacaatggatcagttacgaaatagtgaaaagttagacctgaaaagtcatccagccggtattgttcccattgagatcgaaggttgtgagaacatcggtacagacaaactttgccagcttgataccttccaaaagagagtggctcaggtgattgaacctgcatgccatatttaaa

SEQ ID NO: 21 - DNA, 46 bp, Artificial Sequence
  primer
ctatggatccgcatcatcatcatcatcacagtgataccgcccctgc

SEQ ID NO: 22 - DNA, 6738 bp, Artificial Sequence
  synthetic construct
gagatatacatatgaaatacctgctgccgaccgctgctgctggtctgctgctcctcgctgcccagccggcgatggccatggatatcggaattaattcggatccgcatcatcatcatcatcacagtgataccgcccctgctgggttccagttggaaaaggttgttatcctaagcagacatggcgtacgcgcgccaaccaaaatgacacaaacgatgcgcaacgtcacacctcaccagtggcctgaatggccggtaaaactcggctatatcacgccccgcggtgaacatctgattagcctgatgggcggtttttatcgagagcgctttcagcaacaaggcttattacctaaggataactgtcctacaccagatgccgtgtatgtttgggcagacgtcgatcaacgcacacgtaaaaccggcgaggccttcttagcgggtcttgctccccagtgtgatttagcgatccaccatcagcaaaacattcagcaggccgatccgctgttccatcctgtgaaagccggtatctgttcgatggataaatcacaggcacacgccgccgttgaaaagcaggcaggcacaccgattgagacgctcaatcaacgctatcaagcatctttagcgctgatgagttcggtactcgattttccaaaatccccctattgtcagcagcacaacattggcaaactctgcgatttttcacaggcgatgcctagcaggctggcgataaatgacgacggtaataaagtggctctcgaaggtgccgtgggactttcatcgacgttggctgaaattttcctgctggaacacgctcagggaatgcctaaagtggcttgggggaatattcacactgagcagcaatgggactctctgttaaaattgcataatgcgcagtttgacttgatgtcgcgcacgccctatatcgccaagcataacggtactccactgctgcaaaccatcgcacacgcactgggttccaatatcgcgagtcgcccactgccggatatttcgccagacaataagatcctgtttattgccggtcacgacaccaatattgccaatatttctggcatgctagggatgacatggacacttccgggacagcccgataacaccccgccgggtggggggctggtgtttgaactatggcagaatccagataaccatcagcaatatgtcgcagttaagatgttctatcaaacaatggatcagttacgaaatagtgaaaagttagacctgaaaagtcatccagccggtattgttcccattgagatcgaaggttgtgagaacatcggtacagacaaactttgccagcttgataccttccaaaagagagtggctcaggtgattgaacctgcatgccatatttaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaag

SEQ ID NO: 23 - DNA, 10331 bp, Artificial Sequence
  synthetic construct
aagcttgcatgagcatgtcacagtcgaattctggggtcacgcggtgcttgagggcgaatacggctccatcggtgagtaacctctctcttactaccacggaaacatcactgacgtaaccaggacccggcggcttatccatcatgggaaacaacacctacaaatccgccagaattctctcggaagaatataacctctactactccgtctggtgcgacggtgaccacgagctgtacgatctctcagtaagtgccaaccggttcccgccactatcgtaaaaacaaaaaatctaacaacaccagacggacccctaccaaatgaacaacatctacacccaacaagacaacatccacctcctaagcagacctctatccagcgtgattgatcgtatcgacgctctccttctggttctgaaatcctgcaagggtaacacatgcatccagccgtggcgggtcctccaccccgacgggtccgtagagagcctcaaagatgcactgcaggtgaaatacgattccttttacaccaaccagcccaaggtgtcgtattcagtatgtgaacccgggtacatcattgaggctgaggggccccaggtcggattgcagtatagagatgggctgagttgggaggcgtggacttgacgattccgtcaagtatgagtatgggtacgaataatgagcgttattgctatgtatttttatagatagtttatttatatatcatgactaaacttgagagccatggaatcaatgaaatgacatggcgagtgtagatcacgatagtcatagtagccgaagtgggcggatagccaagaataacaccagaatcagataacaggaacatcacaaccgatcacaccatagataatatccaaagaagtttaaatagccgagacaaagagaatagagacaagatacatggaacaagaaaggtacacccggtagataaaccctgggacgggcccgagtccttacccatagatcaatcccacgggaacaaaaccaaagtcaacaaccaccaccaccattaccacaaccgcatcaatagaaccggtgaaaaatgacaccatcgaatccttcaccctaagtaaagccctgtacgttgcatatcgcttaagcacaaaagtagtagaatagatatgagcccgcacgcgcggccaacgatccaaactagccctgacatcaaagccagcggcgattgcgccatcaagcccccgtctcacttcatagtggaattgcgggtcacctcactgattgactgtctgtctagacacactcacccacgcatgctgtctgtgcccagaacgtggactttggctctgccgagctagaggatcaaatataagtagattggatgtaggcccgtattttttttatttcgtgtgactcggagattttatgcgttgtgttgttgggcggaaaaagaaatatactttctttttgttcttttctttttctctctattgcttgccttggatatcccttgcatacggtcggttgctgattgactaagggtgctgtcttgtgtcactgaactgctgctcaacctctgtctggtattcctgttgtcgtgatggtggggaaacagttcgagttcgaggaccagagggatggcatcgtgcctcccttggaggaaaagaaggtcgtcgatgaggtctataccgataatgatgttgcgtcggaggagattgtcaaggactgggatgataaggaggagggcaagctgcggaggaagtgagtcgtcactgttttcattcactgccatataggttcaagcatatactgactggtatataggatcgatatcatcctcatccccattctcgctctcgctttcttcggcctccagattgatcgcggcaatatcagcgcagctcttacctccactatcaccgaagacctaggtgtcaccacgaaccaaatcaatattggaacccagttgctttcggctggtattgtcatcaccgagatcccgtcaaatattatacttcagcgcatcggtccccaggtctggttgtcggcacagctgatcgcttggggtctggttggcacattccaggcttttgtacagtcgtacccggcgtatctggccacgaggttgttgctggggctgttggagggagggtttattcctggtttgtctggtcgtgcgccttggtctatggtggtagcgctaacaatgggtttggtacaggtgccctgtactatctctcgacatggtataaacgtcctgagacgagtttccggaccactctgttcttctatgggcagatgtttgccggtgcgacctcgagcggccgcttcgaggattgcctgaacattgacattcggcgtccggccgggaccaccgcggactcgaagctgcctgtgctggtctggatctttggcggaggctttgaacttggttcaaaggcgatgtatgatggtacaacgatggtatcatcgtcgatagacaagaacatgcctatcgtgtttgtagcaatgaattatcgcgtgggaggtttcgggttcttgcccggaaaggagatcctggaggacgggtccgcgaacctagggctcctggaccaacgccttgccctgcagtgggttgccgacaacatcgaggcctttggtggagacccggacaaggtgacgatttggggagaatcagcaggagccatttccgtttttgatcagatgatcttgtacgacggaaacatcacttacaaggataagcccttgttccggggggccatcatggactccggtagtgttgttcccgcagaccccgtcgatggggtcaagggacagcaagtatatgatgcggtagtggaatctgcaggctgttcctcttctaacgacaccctagcttgtctgcgtgaactagactacaccgacttcctcaatgcggcaaactccgtgccaggcattttaagctaccattctgtggcgttatcatatgtgcctcgaccggacgggacggcgttgtcggcatcaccggacgttttgggcaaagcagggaaatatgctcgggtcccgttcatcgtgggcgaccaagaggatgaggggaccttattcgccttgtttcagtccaacattacgacgatcgacgaggtggtcgactacctggcctcatacttcttctatgacgctagccgagagcagcttgaagaactagtggccctgtacccagacaccaccacgtacgggtctccgttcaggacaggcgcggccaacaactggtatccgcaatttaagcgattggccgccattctcggcgacttggtcttcaccattacccggcgggcattcctctcgtatgcagaggaaatctcccctgatcttccgaactggtcgtacctggcgacctatgactatggcaccccagttctggggaccttccacggaagtgacctgctgcaggtgttctatgggatcaagccaaactatgcagctagttctagccacacgtactatctgagctttgtgtatacgctggatccgaactccaaccggggggagtacattgagtggccgcagtggaaggaatcgcggcagttgatgaatttcggagcgaacgacgccagtctccttacggatgatttccgcaacgggacatatgagttcatcctgcagaataccgcggcgttccacatctgatgccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcaatgggcgtctctgctgttctacttcctttgtatctcctgtctgggtatgctaagcaccacaatcaaagtctaataaggaccctcccttccgagggcccctgaagctcggactgtgtgggactactgatcgctgactatctgtgcagagtcacctccggactggcagtccccagtgataccgcccctgctgggttccagttggaaaaggttgttatcctaagcagacatggcgtacgcgcgccaaccaaaatgacacaaacgatgcgcaacgtcacacctcaccagtggcctgaatggccggtaaaactcggctatatcacgccccgcggtgaacatctgattagcctgatgggcggtttttatcgagagcgctttcagcaacaaggcttattacctaaggataactgtcctacaccagatgccgtgtatgtttgggcagacgtcgatcaacgcacacgtaaaaccggcgaggccttcttagcgggtcttgctccccagtgtgatttagcgatccaccatcagcaaaacattcagcaggccgatccgctgttccatcctgtgaaagccggtatctgttcgatggataaatcacaggcacacgccgccgttgaaaagcaggcaggcacaccgattgagacgctcaatcaacgctatcaagcatctttagcgctgatgagttcggtactcgattttccaaaatccccctattgtcagcagcacaacattggcaaactctgcgatttttcacaggcgatgcctagcaggctggcgataaatgacgacggtaataaagtggctctcgaaggtgccgtgggactttcatcgacgttggctgaaattttcctgctggaacacgctcagggaatgcctaaagtggcttgggggaatattcacactgagcagcaatgggactctctgttaaaattgcataatgcgcagtttgacttgatgtcgcgcacgccctatatcgccaagcataacggtactccactgctgcaaaccatcgcacacgcactgggttccaatatcgcgagtcgcccactgccggatatttcgccagacaataagatcctgtttattgccggtcacgacaccaatattgccaatatttctggcatgctagggatgacatggacacttccgggacagcccgataacaccccgccgggtggggggctggtgtttgaactatggcagaatccagataaccatcagcaatatgtcgcagttaagatgttctatcaaacaatggatcagttacgaaatagtgaaaagttagacctgaaaagtcatccagccggtattgttcccattgagatcgaaggttgtgagaacatcggtacagacaaactttgccagcttgataccttccaaaagagagtggctcaggtgattgaacctgcatgccatatttagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttaccctgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttccgggtggtcattggccgcctgtgatgccatctgccactaaatccgatcattgatccaccgcccacgagggcgtctttgctttttgcgcggcgtccaggttcaactctctctgcagctccagtccaacgctgactgactagtttacctactggtctgatcggctccatcagagctatggcgttatcccgtgccgttgctgcgcaatcgctatcttgatcgcaaccttgaactcactcttgttttaatagtgatcttggtgacggagtgtcggtgagtgacaaccaacatcgtgcaagggagattgatacggaattgtcgctcccatcatgatgttcttgccggctttgttggccctattcgtgggatcgatgccctcctgtgcagcagcaggtactgctggatgaggagccatcggtctctgcacgcaaacccaacttcctcttcattctcacggatgatcaggatctccggatgaattctccggcgtatatgccgtatacgcaggcgagaatcaaggaaaagggtaccgagttcttgaaccatttcgtcactaccgcgctttgctgtccgtcgcgcgtgagtctttggacgggaagacaggctcataatactaatgtgacggatgtgaacccgccttatggtatggacactgcttcgatcggtcttgattcttcagcgtggttacaattgctaatgcggcataggcggataccccaaattcgtcgctcaaggcttcaacgaaaacttcctccccgtttggctgcagtccgccggttacaatacctactacacggggaagctgttcaactcgcacagtgtcgctacctataacgcgccctttgtgaacggtttcaatggctccgacttcctcctcgacccccacacatattcctactggaatgcgacataccagcgaaaccatgagcctccgcggagttacgagggacaatatactacggatgtgatgaaggagaaggcatcgggattgttggcagatgcgctggacagtgacgcgccattcttcctgacggtcgcgccgatcgcaccgcacacgaacatcgatgtggaggggctgagcggtgcgggtggaccgaagatgacagagccgctgcctgcaccgagacatgcgcatttgtttgctgatgcaaaggtgccgcggacgcctaatttcaatccggacaaggtgtgtgatatcctgacacagtggtggggacgggcactgacaagagtaggattctggtgcggggtggatccaaaccatggaactacagaaccagaccgtcatcgactacgaagaccatctttatcgccagcgtctgcgcactttgcaagccgtcgatgagatggtggatgcgctgatcacgcagctggaagaaagtgggcagatcgacaatacctacatcatttacagtgctgataacggctaccacattggccatcaccgtctaccccccggcaagacaactggctatgaagaggacattcgcgtaccattctacattcgcggacctggcattcctgagggaaagagcgttgaccgtgtaaccacgcacattgacattgcacctacactgttcgagttggctggggttcccttgcgagaggactttgacgggactccgatgcccgtgtcgactagcaagaagacccagtcaagcttgcatgcctgcaggtcgactctagaggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaactcgagcagctg